SEQUENCE LISTING (40 sequences)

SEQ ID NO: 1 - DNA, 16 bp, Artificial Sequence
  Description of Artificial Sequence Synthetic primer
catcagtcgcatgacg

SEQ ID NO: 2 - DNA, 19 bp, Artificial Sequence
  Description of Artificial Sequence Synthetic primer
ggtcactgggagctgacac

SEQ ID NO: 3 - DNA, 22 bp, Artificial Sequence
  Description of Artificial Sequence Synthetic primer
agaaaccttctctctcagccca

SEQ ID NO: 4 - DNA, 19 bp, Artificial Sequence
  Description of Artificial Sequence Synthetic primer
gccctgcagagaacctaca

SEQ ID NO: 5 - DNA, 17 bp, Artificial Sequence
  Description of Artificial Sequence Synthetic primer
cttcatcgctggtgctg

SEQ ID NO: 6 - DNA, 21 bp, Artificial Sequence
  Description of Artificial Sequence Synthetic primer
caggctcttggtccattacaa

SEQ ID NO: 7 - DNA, 25 bp, Artificial Sequence
  Description of Artificial Sequence Synthetic primer
ggtctatatgagaaaccttcgctta

SEQ ID NO: 8 - DNA, 21 bp, Artificial Sequence
  Description of Artificial Sequence Synthetic primer
agccgaagcatctgtaggtct

SEQ ID NO: 9 - DNA, 22 bp, Artificial Sequence
  Description of Artificial Sequence Synthetic primer
aggtctatttgggaaaccttca

SEQ ID NO: 10 - DNA, 22 bp, Artificial Sequence
  Description of Artificial Sequence Synthetic primer
actcatagggtgagtcatggag

SEQ ID NO: 11 - DNA, 19 bp, Artificial Sequence
  Description of Artificial Sequence Synthetic primer
accatgtcgctcatggtca

SEQ ID NO: 12 - DNA, 18 bp, Artificial Sequence
  Description of Artificial Sequence Synthetic primer
cacagggcccatgaggat

SEQ ID NO: 13 - DNA, 20 bp, Artificial Sequence
  Description of Artificial Sequence Synthetic primer
cgtcaccctcccatgatgta

SEQ ID NO: 14 - DNA, 18 bp, Artificial Sequence
  Description of Artificial Sequence Synthetic primer
cacagggcccatgaggat

SEQ ID NO: 15 - DNA, 21 bp, Artificial Sequence
  Description of Artificial Sequence Synthetic primer
cttctccatcagtcgcatgag

SEQ ID NO: 16 - DNA, 19 bp, Artificial Sequence
  Description of Artificial Sequence Synthetic primer
agggtcactgggagctgac

SEQ ID NO: 17 - DNA, 21 bp, Artificial Sequence
  Description of Artificial Sequence Synthetic primer
cttctccatcagtcgcatgaa

SEQ ID NO: 18 - DNA, 19 bp, Artificial Sequence
  Description of Artificial Sequence Synthetic primer
tgcacagagaggggaagta

SEQ ID NO: 19 - DNA, 21 bp, Artificial Sequence
  Description of Artificial Sequence Synthetic primer
cggacactctcacctgtgatg

SEQ ID NO: 20 - DNA, 19 bp, Artificial Sequence
  Description of Artificial Sequence Synthetic primer
accttgtcctgcagctcct

SEQ ID NO: 21 - DNA, 22 bp, Artificial Sequence
  Description of Artificial Sequence Synthetic primer
gaagcatctgtaggttcctcct

SEQ ID NO: 22 - DNA, 18 bp, Artificial Sequence
  Description of Artificial Sequence Synthetic primer
cagctcccggagctccta

SEQ ID NO: 23 - DNA, 20 bp, Artificial Sequence
  Description of Artificial Sequence Synthetic primer
tgacggaaacaagcagtgga

SEQ ID NO: 24 - DNA, 20 bp, Artificial Sequence
  Description of Artificial Sequence Synthetic primer
ccttgtcctgcagctccatc

SEQ ID NO: 25 - DNA, 20 bp, Artificial Sequence
  Description of Artificial Sequence Synthetic primer
tgacggaaacaagcagtgga

SEQ ID NO: 26 - DNA, 19 bp, Artificial Sequence
  Description of Artificial Sequence Synthetic primer
agagaggggacgtttaacc

SEQ ID NO: 27 - DNA, 18 bp, Artificial Sequence
  Description of Artificial Sequence Synthetic primer
tccagagggtcactgggc

SEQ ID NO: 28 - DNA, 21 bp, Artificial Sequence
  Description of Artificial Sequence Synthetic primer
tgagcacttctttctgcacaa

SEQ ID NO: 29 - DNA, 20 bp, Artificial Sequence
  Description of Artificial Sequence Synthetic primer
gtaggtccctgcaaggkcaa

SEQ ID NO: 30 - DNA, 18 bp, Artificial Sequence
  Description of Artificial Sequence Synthetic primer
aacccttcctgtctgccc

SEQ ID NO: 31 - DNA, 18 bp, Artificial Sequence
  Description of Artificial Sequence Synthetic primer
ggaagatgggaacgtggc

SEQ ID NO: 32 - DNA, 20 bp, Artificial Sequence
  Description of Artificial Sequence Synthetic primer
cctgcaatgttggtcagatg

SEQ ID NO: 33 - DNA, 21 bp, Artificial Sequence
  Description of Artificial Sequence Synthetic primer
gagccgacaactcatagggta

SEQ ID NO: 34 - DNA, 16 bp, Artificial Sequence
  Description of Artificial Sequence Synthetic primer
cgctgtggtgcctcgc

SEQ ID NO: 35 - DNA, 21 bp, Artificial Sequence
  Description of Artificial Sequence Synthetic primer
acctgtgaccatgatcaccat

SEQ ID NO: 36 - DNA, 22 bp, Artificial Sequence
  Description of Artificial Sequence Synthetic primer
acatgtgattcttcggtgtcat

SEQ ID NO: 37 - DNA, 21 bp, Artificial Sequence
  Description of Artificial Sequence Synthetic primer
tgtgaaccccgacatctgtac

SEQ ID NO: 38 - DNA, 23 bp, Artificial Sequence
  Description of Artificial Sequence Synthetic primer
ctttccagggttcttcttgctgc

SEQ ID NO: 39 - DNA, 21 bp, Artificial Sequence
  Description of Artificial Sequence Synthetic primer
gaaaacggtgtttcggaatac

SEQ ID NO: 40 - DNA, 17 bp, Artificial Sequence
  Description of Artificial Sequence Synthetic primer
tgcgctgctgagctgag

(19) United States
(12) Patent Application Publication
(10) Pub. No.: US 2015/0056621 A1
(43) Pub. Date: Feb. 26, 2015

(54) PRIMERS, METHODS AND KITS FOR DETECTING KILLER-CELL IMMUNOGLOBULIN-LIKE RECEPTOR ALLELES

(71) Applicant: LIFE TECHNOLOGIES CORPORATION, Carlsbad, CA (US)

(72) Inventors: Lu WANG, Potomac, MD (US); David Dinauer, Fox Point, WI (US)

(21) Appl. No.: 14/477,293

(22) Filed: Sep. 4, 2014

Related U.S. Application Data
(62) Division of application No. 10/595,487, filed on Jul. 22, 2008.
(60) Provisional application No. 60/513,307, filed on Oct. 22, 2003.

(51) Int. Cl. C12Q 1/68

(52) U.S. Cl. CPC C12Q 1/6883; C12Q 2600/16
USPC 435/6.11

(57) ABSTRACT

Embodiments of the present invention describe primer pairs, methods and kits for identifying and/or detecting killer-cell immunoglobulin-like receptor (KIR) alleles. The present primer sets include one or more primer pairs that can produce amplicons specific for an individual KIR allele and that are less than 1000 bp in size. Additionally, the primer sets can target intra-exon and/or extracellular domains of KIR alleles for amplification.

### CROSS-REFERENCE TO RELATED APPLICATIONS

This application is a divisional of U.S. patent application Ser. No. 10/595,487 filed Jul. 22, 2008, which is the National Stage of International Application PCT/US2004/007925 filed Mar. 15, 2004, which claims the benefit of U.S. Provisional Patent Application 60/513,307 filed Oct. 22, 2003, all of which are hereby incorporated by reference in their entirety.

### FIELD OF INVENTION

Embodiments of the present invention relate to primer pairs, primer sets and methods of using the primer pairs and primer sets to identify KIR alleles. Certain embodiments also encompass kits for use with the present primers or methods. More particularly this invention relates to primer pairs, primer sets and methods of identifying KIR alleles by amplification of approximately 1000 bp amplicons from an intra-exon portion of the KIR extracellular domain.

### BACKGROUND OF THE INVENTION

Several types of highly diversified molecules, such as the ABO blood group system, the family of MHC (Major Histocompatibility Complex, called, in humans, HLA-human leukocyte antigen), the family of receptors for the T lymphocyte antigen (TCR) and the family of receptors for the B lymphocyte antigen (BCR) partially characterize immune functions in humans and animals. Each of these different families of molecules, when expressed by an adult individual, constitute an individually specific repertoire that is involved in immune system self or non-self recognition.

Immunologists have more recently identified other specific immune system repertoires, such as the repertoire defined by natural killer-cell immunoglobulin-like receptors (KIRs). The KIR family of natural killer (NK) cell receptors, a family with currently approximately 14 genes and 2 pseudogenes, is highly polymorphic and mimics on natural killer cells the clonotypic expression of TCRs and BCRs on T-cells and B-cells. The diverse members of the KIR family participate in mediating cell-cell recognition by NK cells.

Generally, NK cells exhibit cytotoxic activity following recognition of non-self during cell-cell interactions. Inhibitory KIR family members, whose natural ligands are members of the MHC Class I complex, prevent NK cytotoxicity upon ligand binding. Thus, when a KIR finds its natural ligand on a cell, it recognizes the cell as self. This mechanism suggests that inhibitory KIR receptors play a role in preventing autoimmune reactions. Inhibitory KIRs may also participate in the antigenic incompatibility during allograft or xenograft transplantation.

In in vivo allograft hemopoietic transplantation, scientists have demonstrated the involvement of KIRs in a graft versus leukemia, a positive side effect of allograft transplantation. In patients undergoing hemopoietic transplants, a balance must be achieved between the incidence of graft versus host disease, i.e. where the transplanted cells begin to attack the healthy cells of the recipient, and graft versus leukemia, i.e. where the transplanted cells only attack pathogenic cells. KIRs present on the graft but lacking a matched MHC Class I ligand in the recipient have been implicated in a reduced risk of leukemia relapse in patients receiving bone marrow transplants. Presumably, KIRs contribute to an increase in graft versus leukemia. Although scientists recognize the beneficial effects of graft versus leukemia, currently grafted tissue or cells are not screened for cells that contribute to graft versus leukemia. As KIRs appear to be involved in this process, it would be highly beneficial to be able to screen transplanted tissue or cells in order to determine KIRs that may increase graft versus leukemia.

Effectively typing individual alleles of KIRs in a graft or in a patient is complicated by the degree of complexity in KIR expression. The currently available means used in the medical context do not make it possible to easily and inexpensively document all the presently known KIR repertoires during transplantation. Only the compatibility of the HLA-A, HLA-B, and HLA-DR molecules of the donor and of the recipient are currently checked prior to a grant or transplant. Although systems have been developed in an attempt to establish KIR typing, none of the presently available systems provide a way to type all presently known KIR alleles. Because KIR alleles are closely related, their close homology often makes it impossible to discriminate between alleles without sophisticated procedures such as nucleotide sequencing.

Thus, there continues to be a need for identifying known KIR receptors.

### SUMMARY OF THE INVENTION

In one embodiment a primer pair for identifying a killer-cell immunoglobulin-like receptor allele is described. The primer set consists of primers capable of amplifying all presently known KIR alleles.

According to this primer set amplicons that are less than or 1000 bases in length are amplified from an intra-exon portion of a nucleic acid that encodes for an extracellular portion of a KIR receptor.

In alternative embodiments, amplicon size may vary and amplicons may be less than or 500 or 250 bases in length or greater than or 2000 bases in length.

Based on these primer sets, methods of detecting KIR alleles using the primer sets are described. Kits for carrying out these methods are also provided in some embodiments. These kits can include

instructions for carrying out the methods, one or more reagents useful in carrying out these methods, and one or more primer sets capable of amplifying all presently known KIR alleles.

Objects and advantages of the present invention will become more readily apparent from the following detailed description.

### BRIEF DESCRIPTION OF THE DRAWINGS

FIG. 1 is an image of an electrophoretic gel showing the results of amplifying a sample containing KIR alleles with a primer set described herein with different thermal cyclers.

FIG. 2 is an enlarged image of an electrophoretic gel showing the results of amplifying a sample containing KIR alleles with a primer set described herein.

FIG. 3 is an image of an electrophoretic gel showing the results of amplifying a sample containing KIR alleles with a primer set described herein with different DNA template quantities.

FIG. 4 is an image of an electrophoretic gel showing the results of amplifying a sample containing KIR alleles with a primer set described herein with different DNA polymerase amounts.

FIG. 5 is an image of an electrophoretic gel showing the results of amplifying a sample containing KIR alleles with a primer set described herein with a denaturing temperature in the thermal cycling reaction of 92° C.

FIG. 6 is an image of an electrophoretic gel showing the results of amplifying a sample containing KIR alleles with a primer set described herein with an annealing temperature in the thermal cycling reaction of 61° C.

FIG. 7 is an image of an electrophoretic gel showing the results of amplifying a sample containing KIR alleles with a primer set described herein with an annealing temperature in the thermal cycling reaction of 65° C.

FIG. 8 shows selected IHW panel sample results using a primer set and method described herein.

### DETAILED DESCRIPTION

Described herein are primer sets, methods and kits for detecting one or more killer-cell immunoglobulin-like receptor (KIR) alleles. KIRs, members of the immunoglobulin (Ig) superfamily, mediate the function of natural killer cells in innate immune responses and play an important role in regulating Natural Killer (NK) cell activity. KIR receptors are found on the surface of human NK cells and some T-cell subsets. These receptors recognize Class I MHC molecules expressed on target cells and some of these receptors directly interact with polymorphic HLA-A, -B or -C determinants. For example, inhibitory KIRs use HLA Class I as ligands (Bw4 and Cw epitopes). This recognition helps determine whether the target should be lysed—if the KIR fails to recognize the appropriate ligand, the NK cell becomes cytotoxic.

Some pathogens and tumor infected cells evade the immune system by down-regulating HLA Class I molecules. When HLA Class I is downregulated, cytotoxic T cells no longer have the opportunity to recognize and react to peptides bound to HLA. The immune system can counteract the loss of immunicity caused by down-regulated MHC Class I through the KIR/NK mechanism. This phenomenon, which results in cytotoxicity when NK cells bind to non-self cells, has been described as the “missing-self hypothesis.” KIRs involved in the missing-self hypothesis are inhibitory KIRs. Additionally, KIRs can be activating. However, the ligand is currently unknown for activating KIRs.

KIR diversity in individuals is achieved through many factors, including: allelic variability, gene content; gene copy number and gene expression. Individual KIR genes may be exhibited more than once on one haplotype. Expression of KIRs in individuals varies significantly and individuals may exhibit between 8 and 16 genes/pseudogenes. KIR receptors and alleles are described in the following references, which are hereby incorporated by references. Carrington M, Norman P. The KIR Gene Cluster: Bethesda Md.: National (USA) Library of Medicine, NCBI, 2003 http://www.ncbi.nlm.nih.gov; Hsu K, Liu X, Selvakumar A, Mickelson E, O'Reilly R, Dupont B. The Journal of Immunology, 2002, 169:51 18-5129; Yawata M, Yawata N, Abi-Rached L, Parham P. Critical Reviews in Immunology, 2002, 22:463-482; Gomez-Lozano and Vilches, Tissue Antigen 2002:59:184-1 93; Killer cell immunoglobulin-like receptor (KIR) Nomenclature report, 2002 Steven G. E. Marsh, et al. Human Immunology 64: 648-654; Genotyping of human killer-cell immunoglobulin-like receptor genes by polymerase chain reaction with sequence specific primers: Diverse, Rapidly Evolving Receptors of Innate and Adaptive Immunity; Vilches, C, Parham, P, Annual Review in Immunology, 2002 20:217-251; A structural perspective on MHC class I recognition by killer cell immunoglobulin-like receptors; Boyington, J, Sun, P, Molecular Immunology, 2001 38: 1007-1021; The killer cell immunoglobulin-like receptor (KIR) genomic region: gene-order, haplotypes and allelic polymorphism; Hsu, K, Chida, S, Geraghty, D, Dupont, B, Immunological Reviews, 2002 1 90:40-52; and Structure and function of major histocompatibility complex (MHC) class I specific receptors expressed on human natural killer (NK) cells; Borrego, F, Kabat, J, et al. Molecular Immunology 2001 38:637-660. Sequences of the presently known KIR alleles are reported at the Immuno Polymorphism Database (IPD) website www.ebi.ac.uk/ipd/kir/ and the National Center for Biotechnology Information (NCBI) website—dbMHC, www.ncbi.nlm.nih.gov/mhc/.

One embodiment of a primer set provides a primer set that identifies all of the presently known KIR alleles. Such a primer set can include a plurality of primer pairs such that a majority of the primer pairs are capable of producing an amplicon that is less than or 1000 bases in length from a nucleic acid that encodes a KIR receptor. Amplification of smaller PCR products is more efficient and tolerant of assay variables such as degraded DNA and reduced polymerase activity. Accordingly, the present primer sets, methods and kits exhibit a robustness allowing them to be used with minimal experimentation. In some embodiments, the majority of the primer pairs are capable of producing an amplicon that is less than or 1000 bases in length from an intra-exon portion of a nucleic acid that encodes for a portion of a KIR receptor. In these and other embodiments, one or more of the primer pairs of the majority of the primer pairs are capable of producing an amplicon that is less than or 1000 bases in length from a nucleic acid that encodes for an extracellular portion of a KIR receptor.

In another embodiment, a primer set includes a first primer and second primer that together are capable of producing an amplicon that is less than or 1000 bases in length from an intra-exon portion of a nucleic acid that encodes for an extracellular portion of a KIR receptor. Accordingly, in this embodiment a KIR primer pair targets intra-exon polymorphism such that the gene-specific amplicon sizes do not exceed 1000 bp. As above, amplification of smaller PCR products is more efficient and tolerant of assay variables such as degraded DNA and reduced polymerase activity so that the primer sets, the described methods, and kits, are more robust than previous compositions and methods for determining individual KIR alleles. The primer sets may also contain additional primer pairs that are specific for a desired KIR allele. For example, one or more of such primer pairs can be capable of producing an amplicon that is less than or 1000 bases in length from an intra-exon portion of a nucleic acid that encodes for an extracellular portion of one or more additional KIR receptors. In some embodiments, the primer set contains primer pairs that are capable of identifying all presently known KIR receptors. In these primer sets a majority of the primer pairs in the primer set are capable of producing an amplicon that is less than or 1000 bases in length from an intra-exon portion of a nucleic acid that encodes for an extracellular portion of the KIR receptors.

In any of the above primer sets a majority of the primer pairs can produce an amplicon that is less than or 250 or 500 bases in length. In certain embodiments a majority of the primer pairs are capable of producing an amplicon from about 100, 150, 200 or 250 to about 250, 5Q0, 750 or 1000 bases in length. However, the present primer sets do not require that all the primer pairs produce amplicons from KIR alleles that are less than or 1000 bases in length. As such, one or more primer pairs can be capable of producing an amplicon that is greater than 1000 bases in length. Further, primer pairs are capable of producing an amplicon from an inter-exon of a nucleic acid that encodes for a portion of a KIR receptor.

In some embodiments, none of the primer pairs are capable of producing an amplicon greater than or 2000, 3000, 4000 or 5000 bases in length. In further embodiments, in place of a majority about 25%, 30%, 35%, 40%, 45%, 50%, 55%, 60%, 65%, 75%, 80%, 85%, 90% or 95% of the primer pairs in any of the primer sets described herein can have the recited characteristics.

Suitable intra-exon or extracellular KIR domains that can be amplified by the present primer pairs include those encoded by any one of KIR exons 1-8, and in particular the polymorphic regions of these exons. Different primer pairs can produce amplicons from different KIR exons as desired. As will be apparent to those skilled in the art, the exact sequence of the primers or amplicons produced are not critical to the present primer sets, methods or kits as long as the KIR allele(s) being tested for can be specifically identified. As such, the present primers will generally target KIR allele sequences that are unique to the specific allele and distinguish one KIR allele from the others. It will also be apparent that with some KIR alleles such distinguishing sequences can be found in many different portions of the KIR gene. Accordingly, the primer pairs typically target polymorphism in the extracellular domains of the KIR receptors based on the fact that more nonsynonymous mutation events occur in these regions compared to the intra cellular and transmembrane regions of each gene. Therefore the primer sets, method and kits can provide high resolution or allele level genotyping methods that primarily target polymorphism in the extra cellular domains of the KIR genes.

The individual primers in the primer sets can be of any length, for example ranging from 5 nucleotides to several hundred. Preferably, the primer oligonucleotides will have a length of greater than 10 nucleotides, and more preferably, a length of from 1 2-50 nucleotides, such as 12-25 or 15-20 nucleotides. The primer oligonucleotides can also be chosen to have a desired melting temperature, such as 40 to 80° C., 50 to 70° C., 55 to 65° C., or 60° C. The length of the primer is sufficient to permit the primer oligonucleotide to hybridize to the target molecule. The sequence of the primer oligonucleotide is selected such that it is complementary to a predetermined sequence of the target molecule. The 3′ terminus of the primers in the primer sets are capable of being extended by a nucleic acid polymerase under appropriate conditions. The present primers can be used in any method where nucleic acid primers find utility. For example, the primers are readily applicable to RT PCR of KIR mRNA for expression analysis because they may target exon regions. The present primer pairs can also be used individually to identify a single KIR allele, as desired. The present primers can also be extended to, as yet, unknown KIR alleles.

One example of an assay where the present primer pairs find use include detection assays or methods for identifying a KIR allele in a sample having, or suspected of having, a KIR allele. In such an assay, generally, the sample will be contacted with the primer set under conditions such that the primer pair will amplify the KIR allele for which the primer pair is specific, if that allele is present in the sample. The presence or absence of the amplicon can then be determined or detected by standard techniques, such as separation techniques including electrophoresis, chromatography (including HPLC and denaturing-HPLC), or the like. Exemplary techniques for performing these assays are described in the examples section. As will be recognized by the skilled artisan, the production of a specific amplicon will indicate the presence of a specific KIR allele in a sample. Accordingly, the presence or absence of an amplicon can be correlated with the presence or absence of the specific KIR allele in the sample. The sample to be detected can be obtained from any suitable source or by any suitable technique.

Typically, the nucleic acid amplification or extension of the KIR alleles involves mixing a target nucleic acid with a “master mix” containing the reaction components for performing the amplification reaction. This reaction mixture is then subjected to temperature conditions that allow for the amplification of the target KIR. The reaction components in the master mix can include a buffer which regulates the pH of the reaction mixture; one or more of the four deoxynucleotides (dATP, dCTP, dGTP, dTTP—preferably present in equal concentrations), which provide the energy and nucleosides necessary for the synthesis of DNA; primers or primer pairs that bind to the DNA template in order to facilitate the initiation of DNA synthesis; and a DNA polymerase that adds the deoxynucleotides to the complementary DNA strand being synthesized. The polymerase used in the present methods and kits is not particularly limited, and any suitable polymerase can be used. Examples of suitable polymerase include thermostable polymerase enzymes, such as the Taq polymerase. Preferred polymerases exhibit low error rates during strand synthesis.

A typical thermal cycling reaction has a temperature profile that involves an initial ramp up to a predetermined, target denaturation temperature which is high enough to separate the double-stranded target DNA into single strands. Generally, the target denaturation temperature of the thermal cycling reaction is approximately 91-97° C. and the reaction is held at this temperature for a time period ranging between 20 seconds to two minutes. Then, the temperature of the reaction mixture is lowered to a target annealing temperature which allows the primers to anneal or hybridize to the single strands of DNA. Annealing temperatures can vary greatly depending upon the primers and target DNA used. Individual KIR alleles may exhibit individual or equivalent annealing temperatures. Generally, annealing temperatures range from 37° C. to 55° C. depending upon the application. Next, the temperature of the reaction mixture is raised to a target extension temperature to promote the synthesis of extension products. The extension temperature is generally held for approximately two minutes and occurs at a temperature range from 50° C. to 72° C. This completes one cycle of the thermal cycling reaction. The next cycle then starts by raising the temperature of the reaction mixture to the denaturation temperature. Typically, the cycle is repeated 25 to 35 times to provide the desired quantity of DNA. As will be understood by the skilled artisan, the above description of the thermal cycling reaction is provided for illustration only, and accordingly, the temperatures, times and cycle number can vary depending upon the nature of the thermal cycling reaction and application.

The present assays and methods can be performed using a single primer pair specific for a single KIR allele or can use a set of primers that have specificity for more than one KIR allele. In some embodiments, different primer sets are contained within different amplification vessels, such as different wells of a multi-well plate, so that only a single primer set specific for a single KIR allele is present in an individual amplification vessel. Such a configuration simplifies use and interpretation of the assay results. However, the present assays can use multiplex configurations where two, three or more primer pairs that are specific for different KIR alleles can be used in the same reaction vessel and one or more reaction vessels can be utilized. Amplification reactions using different primer pairs can be run in parallel, simultaneously or subsequent to one another, as desired. In some embodiments, all of the primer pairs required to identify all presently known KIR alleles can be contained within the same reaction vessel. In these multiplex assays, typically the primer pairs will be designed so that the resulting amplicons that are specific for a single KIR allele can be distinguished from one another. For example, the amplicons for the different KIR alleles can all have different lengths, or the primer pairs or amplicons can have distinct labels or be distinctly labeled.

In some embodiments, the present KIR primer sets, methods and kits can use a standard sequence specific primer technique. In a non-limiting example, twenty pre-aliquoted primer pairs, plus an optional internal control as well as an optional negative contamination control, can be used to identify all presently known KIR alleles. The methods and kits can also include a nucleic acid amplification buffer, with or without a polymerase, which in some instances will be aliquoted in per test volumes. The present assays can also use positive and negative controls to help verify results.

In some embodiments, the present primer sets, assays and kits can identify the presence of all presently known alleles of KIR genes: 2DL1, 2DL2, 2DL3, 2DL4, 2DL5, 2DP1, 2DS1, 2DS2, 2DS3, 2DS4, 2DS5, 3DL1, 3DL2, 3DL3, 3DP1, and 3DS1, as well as the more recently described variants such as the 2DS4 homolog KIR1 D. Primer sets including primer pairs specific for one, two, three, four, five, six, seven, eight, nine, ten, eleven, twelve, thirteen or more KIR alleles can be designed for producing an amplicon that is less than or 1000 bases in length from an intra-exon portion of a nucleic acid that encodes for an extracellular portion of a KIR receptor. Other primer pairs that produce amplicons in excess of 1000 bases in length can be used in conjunction, such as in parallel, with primer pairs producing the smaller amplicon. All primer mixes can target polymorphism in the extracellular domains of KIRs. All primer mixes can also contain a distinct internal control primer set to ensure proper assay performance. The assay can be completed in less than 3 hours when starting with genomic DNA and can follow the four procedure steps typical of the sequence specific priming (SSP) technology: (1) prepare a master mix with sample DNA, (2) dispense the master mix into the SSP mixes, (3) thermal cycle, and (4) analyze the amplicons by gel electrophoresis. The SSP assay can provide reliable and unambiguous detection of all presently known KIR alleles in a simple and rapid format.

The present primer sets, methods and kits can be used to define or identify KIR haplotypes, genotypes and polymorphic variation in an individual or in different populations. Some embodiments can also be used to identify KIR compatible and incompatible stem cell transplant donor-recipient pairs as well as study if KIR mismatching between donor and recipient correlates with KIR epitope mismatch predicted by HLA. Some embodiments can be used to determine the effect of KIR receptors on post-transplant complications. KIR has also been implicated in association with a wide variety of diseases including HIV, rheumatoid vasculitis, psoriatic arthritis, as well as playing a role in bone marrow transplantation. Regarding the association of KIR with bone marrow research and transplantation, NK Cells have been implicated in promotion of engraftment and mediation of graft versus leukemia (GVL). More recently, it has been suggested that the graft versus leukemia effects are a result of donor-recipient HLA epitope mismatching for KIR. Consistent with “missing self hypothesis”—if transplant recipient lacks HLA Class I determinant recognized by the donor KIR, the NK cells to become cytotoxic towards tumor cells, reducing the risk of relapse. Therefore a mismatch of KIR ligands between host and donor may be preferable to promote GVL. Accordingly, the present primers, methods and kits can be used for research and clinical applications for any KIR associated disease, disorder, condition or phenomenon.

Any or all of the present primers can be labeled with a detectable moiety, if desired, to facilitate detection. When present, the detectable moiety is not particularly limited. Suitable examples of detectable labels include fluorescent molecules, beads, polymeric beads, fluorescent polymeric beads and molecular weight markers. Polymeric beads can be made of any suitable polymer including latex or polystyrene.

Certain embodiments also provide arrays of individual primers, primer pairs and primer sets that are contained within distinct, defined locations on a support. Each defined, distinct area of the array will typically have a plurality of the same primers. In some embodiments, the primers will be physically attached to the support in the defined location. The primers can also be contained within a well of the support. As used herein the term well is used solely for convenience and is not intended to be limiting. For example, a well can include any structure that serves to hold the nucleic acid primers in the defined, distinct area on the solid support. Non-limiting example of wells include depressions, grooves, walled surroundings and the like. In some of the arrays, the primers at different location can have the same probing regions or consist of the same molecule. This embodiment is useful when testing whether nucleic acids from variety of sources contain the same target sequences. The arrays can also have primers with one or different primer regions at different location within the array. This embodiment can be useful where nucleic acids from a single source are assayed for a variety of target sequences. Combinations of these array configurations are also provided where some of the primers in the defined locations contain the same primer regions whereas other locations contain primers with primer regions that are specific for different targets.

Any suitable support can be used for the present arrays, such as glass or plastic, either of which can be treated or untreated to help bind, or prevent adhesion of, individual primers, primer pairs or primer sets. In some embodiments, the support will be a multi-well plate so that the primers need not be bound to the support and can be free in solution. Such arrays can be used for automated or high volume assays for target nucleic acid sequences.

Although the present primers generally utilize the five standard nucleotides (A, C, G, T and U) in their nucleotide sequences, the identity of the nucleotides or nucleic acids are not so limited. Non-standard nucleotides and nucleotide analogs, such as peptide nucleic acids and locked nucleic acids can be used as desired. Several nucleotide analogs are known in the art (e.g., see, in Rawls, C & E News Jun. 2, 1997 page 35; in Brown, Molecular Biology LabFax, BIOS Scientific Publishers Limited; Information Press Ltd, Oxford, UK, 1 991). In addition, the bases in a primer sequence may be joined by a linkage other than a phosphodiester bond, so long as the bond does not interfere with hybridization with KIR alleles. Nucleotide analogs can include any of the known base analogs of DNA and RNA such as, but not limited to, 4-acetylcytosine, 8-hydroxy-N-6-methyladenosin-e, aziridinylcytosine, pseudoisocytosine, 5-(carboxyhydroxylmethyl) uracil, 5-fluorouracil, 5-bromouracil, 5-carboxymethylaminomethyl-2-thiou-racil, 5-carboxymethylaminomethyluracil, dihydrouracil, hypoxanthine, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiou-racil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, orotic acid, 2,6-diaminopurine and the AEGIS™ bases isoC and isoG. As such, the individual primers, primer pairs and primer sets can contain DNA, RNA, analogs thereof or mixtures (chimeras) of these components.

Universal nucleotides can also be used in the present primers. As used herein, universal nucleotide, base, nucleoside or the like, refers to a molecule that can bind to two or more, i.e., 3, 4, or all 5, naturally occurring bases in a relatively indiscriminate or non-preferential manner. In some embodiments, the universal base can bind to all of the naturally occurring bases in this manner, such as 2′-deoxyinosine (inosine). For example, the universal base can bind all of the naturally occurring bases with equal affinity, such as 3-nitropyrrole 2′-deoxynucleoside (3-nitropyrrole) and those disclosed in U.S. Pat. Nos. 5,438,131 and 5,681,947. Generally, when the base is “universal” for only a subset of the natural bases, that subset will generally either be purines (adenine or guanine) or pyrimidines (cytosine, thymine or uracil). Examples of nucleotides that can be considered universal for purines are known as the “K” base (N6-methoxy-2,6-diaminopurine), as discussed in Bergstrom et al., Nucleic Acids Res. 25:1935 (1997) and pyrimidines are known as the “P” base (6H,8H-3,4-dihydropyrimido[4,5-c][1,2]oxazin-7-one), as discussed in Bergstrom et al., supra, and U.S. Pat. No. 6,313,286. Other suitable universal nucleotides include 5-nitroindole (5-nitroindole 2′-deoxynucleoside), 4-nitroindole (4-nitroindole 2′-deoxynucleoside), 6-nitroindole (6-nitroindole 2′-deoxynucleoside) or 2′-deoxynebularine. A partial order of duplex stability has been found as follows: 5-nitroindole>4-nitroindole>6-nitroindole>3-nitropyrrole. When used, such universal bases can be placed in polymorphic positions, for example those that are not required to specifically identify an allele. Combinations of these universal bases can also be used as desired.

Certain embodiments also provide kits for carrying out the methods described herein. In one embodiment, the kit is made up of one or more of the described primer pairs or primer sets with instructions for carrying out any of the methods described herein. The instructions can be provided in any intelligible form through a tangible medium, such as printed on paper, computer readable media, or the like. A plurality of each primer pair or primer set can be provided in a separate container for easy aliquoting. The present kits can also include one or more reagents, buffers, hybridization media, salts, nucleic acids, controls, nucleotides, labels, molecular weight markers, enzymes, solid supports, dyes, chromatography reagents and equipment and/or disposable lab equipment, such as multi-well plates (including 96 and 384 well plates), in order to readily facilitate implementation of the present methods. Such additional components can be packaged together or separately as desired. Solid supports can include beads and the like whereas molecular weight markers can include conjugatable markers, for example biotin and streptavidin or the like. Enzymes that can be included in the present kits include DNA polymerases and the like. Examples of preferred kit components can be found in the description above and in the following examples.

In one embodiment of a kit, the kit contains sequence specific primers to identify the common forms of the following KIR genes: 2DL1, 2DL2, 2DL3, 3DL1, 3DL2, 2DS1, 2DS2, 2DS3, 2DS4, 2DS5, 3DS1, 2DL4, 2DL5, 3DL3, 3DP1, 2DP1. The primer mixes of the kit are present in trays that function under universal reaction conditions, although reaction conditions can differ. Each kit contains 12 tests and 3 or 4 tests per tray are possible. Exemplary, but non-limiting, primer sets are described in Tables 2 and 3. This invention is further illustrated by the following non-limiting examples. These examples demonstrate the present primer sets, methods and kits provide reliable and unambiguous detection of all presently known KIR genes in a simple, fast and cost-effective format.

### EXAMPLES

For KIR genotyping of a single individual, the following protocol was used in the present examples, unless otherwise indicated.

Step 1: Make Mastermix solution (dNTP's, PCR-buffer, MgCl₂, Ficoll, Loading dye)

Step 2: Vortex thoroughly.

Step 3: Dispense 8 μl of the Mastermix solution in each well. Add 1.2 μl Taq Polymerase (5U/μl); 3 μl water; 1.25 μl DNA (at 75-1 25 ng/μl) to the Mastermix solution. Total final reaction volume in each well is 1 3 μl.

Step 4: Seal the tray. Make sure that the DNA-Mastermix-Taq solution has settled completely by gently tapping the tray on the working bench.

Step 5: Cycle on a thermocycler: 95° C. 2 min followed by 30 cycles of 94° C. 20 s, 63° C. 20 s, 72° C. 1 m30 s.

Step 6: Separate the amplicons using 2% agarose gel electrophoresis of 8 μl of the reaction at 150 V for 20-25 minutes.

In all examples, the IHW KIR DNA samples were ordered from IHWG (International Histocompatability Working Group) at the Fred Hutchinson Cancer Research Center in Seattle, Wash. The primer sets used in the example are set forth in Table 2 or Table 3.

### Example 1

As can be seen from FIG. 2, twenty primer mixes identify the presence and absence of all presently known alleles of the KIR genes. The assay identifies 2DL1, 2DL2, 2DL3, 2DL4, 2DL5, 2DP1, 2DS1, 2DS2, 2DS3, 2DS4, 2DS5, 3DL1, 3DL2, 3DL3, 3DP1, and 3DS1 as measured against the sequence alignment resources found at IPD-KIR Sequence Database (http://www.ebi.ac.uk/ipd/kir/) and dbMHC (http://www.ncbi.nlm.nih.gov/mhc/). Each master mix contains a distinct internal control primer set to ensure proper assay performance (Tables 2 and 3 and Figures). A DNA size marker is used to demonstrate relative amplicon sizes. This assay discriminates the more recently described KIR variants 2DL5A and 2DL5B; 2DS4*00101/00101/002 and 2DS4*003; and 3DP1*001/002 and 3DP1*00301/00302. The allele information gained from the reaction may be used to deduce presently known KIR haplotypes.

Functionality of the primer mixes was challenged by testing with a range of variables similar to what may be encountered in routine laboratory use. Table 1 shows the parameters, ranges, and acceptance criteria used with the primer mixes. The parameters show a potential for conservation of sample and reagents.

TABLE 1
Validation Results

| | |
|---|---|
| Parameters | Criteria |

| | | |
|---|---|---|
| DNA template | Higher and | No false positive |
| concentration | Lower and | No false negative or |
| | | failed reactions |
| Taq amount | Higher and | No false positive |
| | Lower and | No band dropout |
| Thermal cycler | Denaturing temp. lower | No band dropout |
| temperature | than 95° C. | |
| | Annealing temp. higher | No band dropout |
| | than 63° C. | |
| | Annealing temp. lower | No false positive |
| | than 63° C. | |

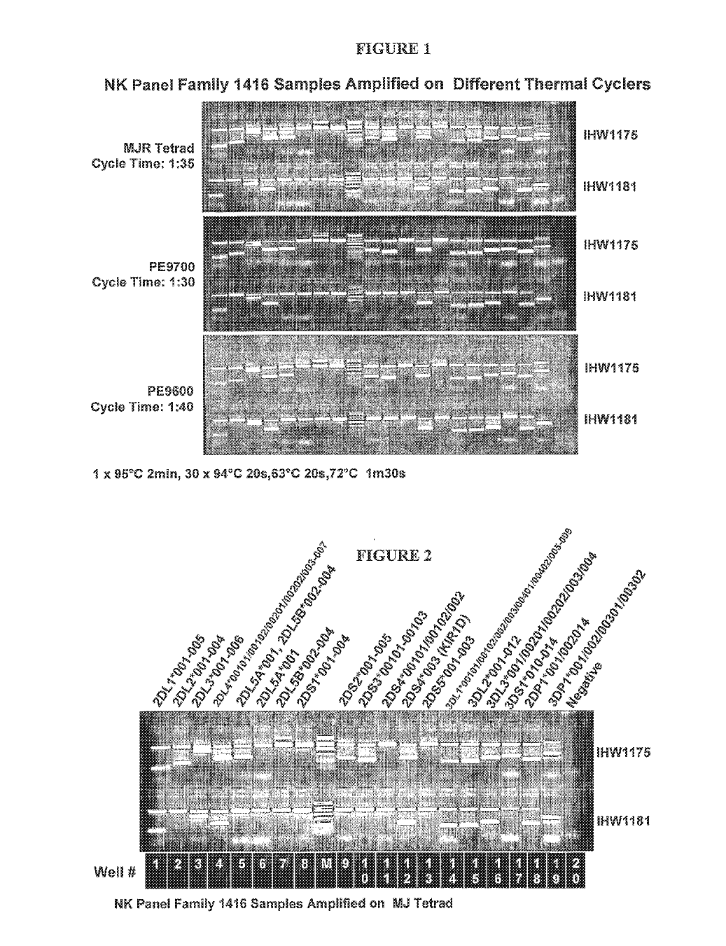

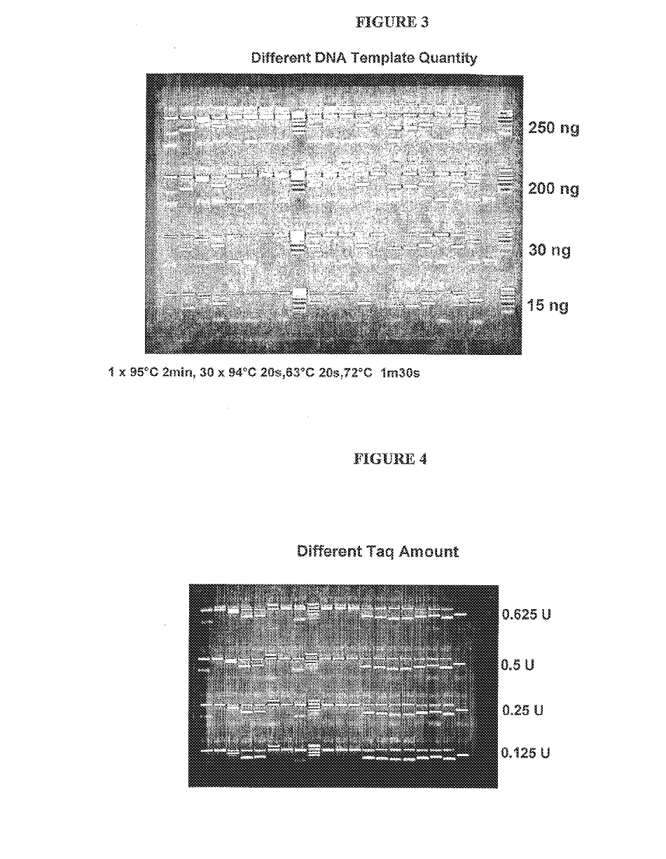

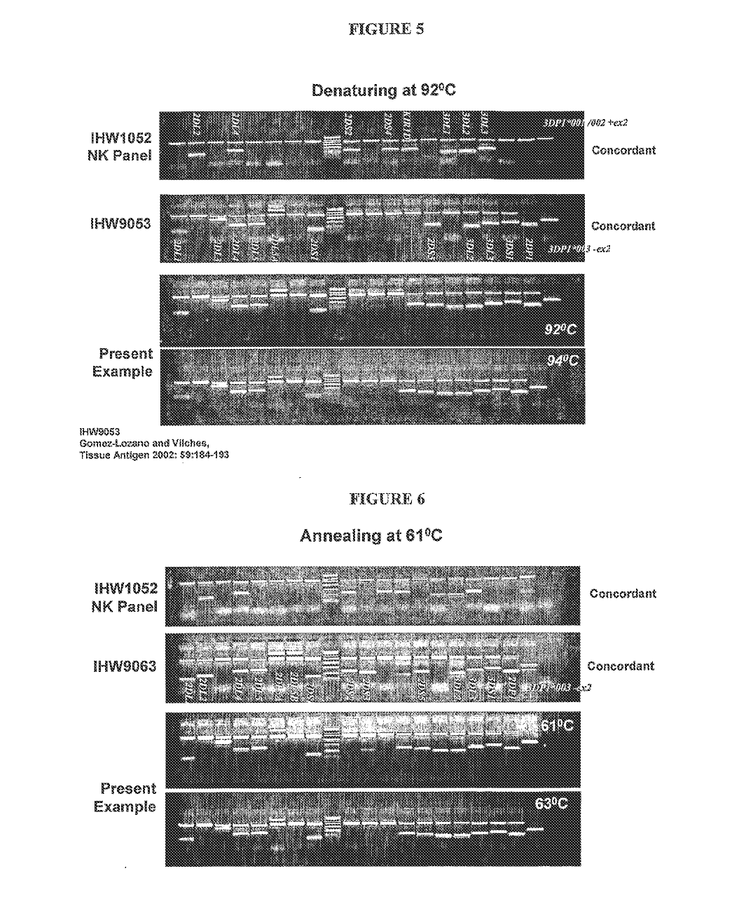

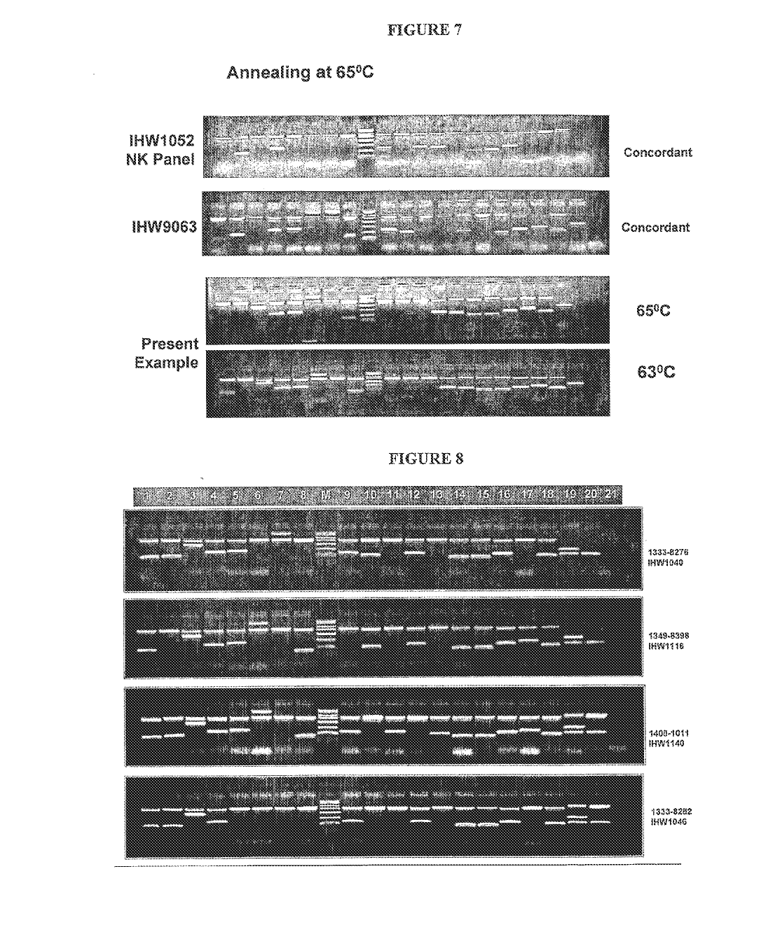

The results discussed in Table 1 are shown in FIGS. 1 and 3-7. Lane assignments for these figures correspond to the wells and primers shown in Tables 2, 3 and/or FIG. 2. Table 2 sets out the exact primer sequences used in the example. Positive results for a given allele are indicated by the presence of multiple bands per sample, as one band corresponds to an internal control. As can be seen from FIG. 1, the results for two subsets of KIR DNA (IHW 1 175 and IHW 1 181) are consistent for all three thermal cyclers used. FIG. 3 illustrates that different DNA template quantities, ranging from 15 ng to 250 ng all provided the same positive results. FIG. 4 demonstrates that differing polymerase amounts also provided consistent allele identification. FIGS. 5-7 demonstrate successful allele identification at different thermocycling annealing and denaturing temperatures. These figures demonstrate that the present primer sets and methods are: specific as they produce the correct amplicon size, robust because specific and abundant amplicons exist at varied conditions, and sensitive relative to template amount.

FIG. 8 shows selected IHW panel sample results. FIG. 8 demonstrates that DNA samples encoding different KIR alleles can be successfully identified using the primers of Table 2.

In the above example, 800 bp internal controls were used (genbank acc#AF442818 C-reactive protein gene—the primer locations for the 800 bp internal control were CRP05 (5′)-18649-18667; CRP06 (3′)-19450-19430; CRP07 (5′)-18642-18663 and CRP08 (3′)-19448-19427), as well as 200 bp internal controls (genbank acc# J04038 Human glyceraldehyde-3-phosphate dehydrogenase (GAPDH) gene—the primer locations for the 200 bp internal control were 5′GPDH-61 9-638; and 3′SGPDH-815-796).

The present primers and kits can have any or all of the components described herein. Likewise, the present methods can be carried out by performing any of the steps described herein, either alone or in various combinations. One skilled in the art will recognize that all embodiments are capable of use with all other appropriate embodiments described herein. Additionally, one skilled in the art will realize that certain embodiments also encompass variations of the present primers, configurations and methods that specifically exclude one or more of the components or steps described herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, particular embodiments encompass not only the entire group listed as a whole, but each member of the group individually and all possible subgroups of the main group. Accordingly, for all purposes, certain embodiments encompass not only the main group, but also the main group absent one or more of the group members. Individual embodiments also envisage the explicit exclusion of one or more of any of the group members.

All references, patents and publications disclosed herein are specifically incorporated by reference thereto. Unless otherwise specified, a or an means one or more. While preferred embodiments have been illustrated and described, it should be understood that changes and modifications can be made therein in accordance with ordinary skill in the art without departing from the invention in its broader aspects as described herein. The broader aspects of the present invention are defined in the following claims.

TABLE 2

| | | | Sense | | Anitsense | | | |
|---|---|---|---|---|---|---|---|---|
| | | | Primer | | Primer | | Internal | App. PCR |
| Well | KIR Allele | | 3′ end | | 3′ end | Exon | Control | product |
| # | Specificity | Sense Primer | location | Antisense Primer | location | Target | size (bp) | size (bp) |
| 1 | 2DL1*001-005 | Seq ID 1-CATCAGTCGCATGACG | 558 | Seq ID 2-GGTCACTGGGAGCTGACAC | 616 | ex4 | 800 | 95 |
| 2 | 2DL2*001-004 | Seq ID 3-AGAAACCTTCTCTCTCAGCCCA | 686 | Seq ID 4-GCCCTGCAGAGAACCTACA | 790 | ex5 | 800 | 145 |
| 3 | 2DL3*001-006 | Seq ID 5-CTTCATCCTGGTGCT | 1094 | Seq ID 6-CAGGCTCTTGGTCCATTACAA | 1112 | ex7-8 | 800 | 455 |
| 4 | 2DL4*00101/00102/ | Seq ID 7-GGTCTATATGAGAAACCTTCGCTTA | 679 | Seq ID 8-AGCCGAAGCATCTGTAGGTCT | 866 | ex5 | 800 | 230 |
| | 00201/00202 | | | | | | | |
| 5 | 2DL5A*001,2DL58*002-004 | Seq IDS 9-AGGTCTATTTGGGAAACCTTCA | 675 | Seq ID 10-ACTCATAGGGTGAGTCATGGAG | 889 | ex5 | 800 | 257 |
| 6 | 2DL5A*001 | Seq ID 11-ACCATGTCGCTCATGGTCA | 15 | Seq ID 12-CACAGGGCCCATGAGGAT | 238 | ex1-3 | 800 | 1753 |
| 7 | 2DL58*002-003 | Seq ID 13-CGTCACCCTCCCATGATGTA | 5′UT | Seq ID 14-CACAGGGCCCATGAGGAT | 238 | ex1-3 | 800 | 1893 |
| 8 | 2DS1*001-004 | Seq ID 15-CTTCTCCATCAGTCGCATGAG | 557 | Seq ID 16-AGGGTCACTGGGAGCTGAC | 616 | ex4 | 800 | 140 |
| | | Seq ID 17-CTTCTCCATCAGTCGCATGAA | 577 | | | | 800 | |
| 9 | 2DS2*001-005 | Seq ID 18-TGCACAGAGAGGGGAAGTA | 482 | Seq ID 19-CGGACATCTCACCTGTGATG | 648 | ex4 | 800 | 207 |
| 10 | 2DS3*00101-00103 | Seq ID 20-ACCTTGTCCTGCAGCTCCT | 739 | Seq ID 21-GAAGCATCTGTAGGTTCCTCCT | 861 | ex5 | 800 | 162 |
| l1 | 2DS4*00101/100102/002 | Seq ID 22-CAGCTCCCGGAGCTCCTA | 749 | Seq ID 23-TGACGGAAACAAGCAGTGGA | 927 | ex5 | 800 | 215 |
| 12 | 2DS4*003(KIR1D) | Seq ID 24-CCTTGTCCTGCAGCTCCATC | 763 | Seq ID 25-TGACGGAAACAAGCAGTGGA | 927 | ex5 | 800 | 200 |
| 13 | 2DS5*001-003 | Seq ID 26-AGAGAGGGGACGTTTAACC | 487 | Seq ID 27-TCCAGAGGGTCACTGGGC | 624 | ex4 | 800 | 179 |
| 14 | 3DL1*00101/00102/002/003/ | Seq ID 28-TGAGCACTTCTTTCTGCACAA | 470 | Seq ID 29-GTAGGTCCCTGCAAGGKCAA | 560 | ex4 | 800 | 129 |
| | 00401/004021005-009 | | | | | | | |
| 15 | 3DL2*001-012 | Seq ID 30-AACCCTTCCTGTCTGCCC | 100 | Seq ID 31-GGAAGATGGGAACGTGGC | 197 | ex3 | 800 | 133 |
| 16 | 3DL3*001/00201/00202/ | Seq ID 32-CCTCAATGTTGGTCAGATG | 442 | Seq ID 33-GAGCCGACAACTCATAGGGTA | 605 | ex4 | 800 | 203 |
| | 003/004 | | | | | | | |
| 17 | 3DS1*010-014 | Seq ID 34-CGCTGTGGTGCCTCGC | 123 | Seq ID 35-ACCTGTGACCAGATCACCAT | 337 | ex3 | 800 | 250 |
| 18 | 2DP1*001/002 | Seq ID 36-ACATGTGATTCTTCGGTGTCAT | 150 | Seq ID 37-TGTGAACCCCGACATCTGTAC | 276 | ex3 | 800 | 171 |
| 19 | 3DP1*0011002 | Seq ID 38-CTTTCCAGGGTTCTTCTTGCTGC | 49 | Seq ID 39-GAAAACGGTGTTTCGGAATAC | 223 | ex2-3 | 200 | 975 |
| | 3DP1*00301/00302 | Seq ID 40-TGCGCTGCTGAGCTGAG | 5′UT | | | ex1-3 | | 344 |
| 20 | Negative Control | | | | | | NONE | 200 |
| | | | | | | | | 800 |

TABLE 3

| | | | Sense | | Antisense | | |
|---|---|---|---|---|---|---|---|
| | | Sense | primer | Antisense | primer | App. Internal | App. PCR |
| | | primer | 3′ end | primer | 3′ end | control size | product |
| Well # | KIR Allele Specificity | 3′ end | location | 3′ end | location | (bp) | size (bp) |
| 1 | 2DL1*001-005 | GAA | 453 | GCG | 557 | 800 | 145 |
| 2 | 2DL2*001-004 | CCA | 686 | ACA | 790 | 800 | 145 |
| 3 | 2DL3*001-006 | CTG | 1094 | CAA | 1112 | 800 | 455 |
| 4 | 2DL4*00101/00102/00201/00202/003-007 | TTA | 679 | TCT | 866 | 800 | 230 |
| 5 | 2DL5A*001, 2DL5B*002-004 | TCA | 675 | GAG | 889 | 800 | 257 |
| 6 | 2DL5A*001 | TCA | 16 | GAT | 238 | 800 | 1753 |
| 7 | 2DL5B*002-004 | GTA | 5′UT | GAT | 238 | 800 | 1893 |
| 8 | 2DS1*001 | GAG | 557 | GAC | 618 | 800 | 100 |
| | 2DS1*002-004 | GAA | 557 | | | | |
| 9 | 2DS2*001-005 | GTA | 482 | ATG | 648 | 800 | 207 |
| 10 | 2DS3*00101-00103 | CCT | 739 | CCT | 861 | 800 | 162 |
| 11 | 2DS4*00101/00102/002 | CTA | 749 | GGA | 927 | 800 | 215 |
| 12 | 2DS4*003 | ATC | 763 | GGA | 927 | 800 | 200 |
| 13 | 2DS5*001-003 | ACC | 487 | GGC | 624 | 800 | 179 |
| 14 | 3DL1*00101/00102/002/003/00401/00402/005-009 | CAA | 470 | CAA | 560 | 800 | 129 |
| 15 | 3DL2*001-012 | CCC | 100 | GGC | 197 | 800 | 133 |
| 16 | 3DL3*001/00201/00202/003/004 | ATG | 442 | GTA | 605 | 800 | 203 |
| 17 | 3DS1*010-014 | CGC | 123 | CAT | 337 | 800 | 250 |
| 18 | 2DP1*001/002 | CAT | 150 | TAC | 276 | 800 | 171 |
| 19 | 3DP1*001/002 | TGC | 49 | TAC | 223 | 200 | 975 |
| | 3DP1*00301/00302 | GAG | 5′UT | | | | 344 |
| 20 | 3DP1*001/002 | TGC | 49 | TAC | 223 | 200 | 975 |
| 21 | Negative Control | | | | | NONE | 200 |
| | | | | | | | 800 |

The three nucleotide sequence given in the table is the sequence of the last three nucleotides of the sense and antisense primers which together determine the allele specificity of each primer mix. The 3′ end sequence of the antisense primer should be read in reverse direction complementary to the sense sequence.

The location of the last base of each primer is given in the table. The numbers correspond to the nucleotide number, not the amino acid codon number. The location of the first nucleotide corresponds to the beginning of the first codon.

What is claimed is:

1. A primer set for identifying a killer-cell
immunoglobulin-like receptor (KIR) allele, comprising:
a first primer pair that comprises a first primer and second primer capable of producing an amplicon that is less than or 1000 bases in length from an intra-exon portion of a nucleic acid that encodes for an extracellular portion of a KIR.

2. The primer set of claim 1, further comprising:
one or more additional primer pairs that comprise a first primer and second primer capable of producing an amplicon that is less than or 1000 bases in length from an intra-exon portion of a nucleic acid that encodes for an extracellular portion of one or more additional KIRs.

3. The primer set of claim 1 wherein the primer set comprises primer pairs that are capable of identifying all presently known KIRs.

4. The primer set of claim 3 wherein a majority of the primer pairs comprise primers that are capable of producing an amplicon that is less than or 1000 bases in length from an intra-exon portion of a nucleic acid that encodes for an extracellular portion of the KIR.

5. A primer set for identifying all of the presently known KIR alleles comprising:
a plurality of primer pairs that are capable of identifying all presently known KIR alleles, wherein a majority of the primer pairs are capable of producing an amplicon that is less than or 1000 bases in length from a nucleic acid that encodes a KIR.

6. The primer set of claim 5 wherein one or more of the primer pairs of the majority of the primer pairs are capable of producing an amplicon that is less than or 1000 bases in length from a nucleic acid that encodes for an extracellular portion of a KIR.

7. The primer set of claim 6 wherein one or more of the primer pairs of the majority of the primer pairs are capable of producing an amplicon that is less than or 1000 bases in length from an intra-exon portion of a nucleic acid encoding for an extracellular portion of a KIR.

8. The primer set of claim 5 wherein a majority of the primer pairs are capable of producing an amplicon that less than or 500 bases in length.

9. The primer set of claim 8 wherein a majority of the primer pairs are capable of producing an amplicon that less than or 250 bases in length.

10. The primer set of claim 5, wherein a majority of the primer pairs are capable of producing an amplicon from 150 to 1000 bases in length.

11. The primer set of claim 5 wherein one or more of the primer pairs of the majority of the primer pairs are capable of producing an amplicon that is less than or 1000 bases in length from an intra-exon portion of a nucleic acid that encodes for a portion of a KIR.

12. The primer set of claim 7 wherein the intra-exon or extracellular portion of the KIR receptor is encoded by any one of KIR exons 1-8.

13. The primer set of claim 5 wherein one or more primer pairs are capable of producing an amplicon that is greater than 1000 bases in length.

14. The primer set of claim 5, wherein none of the primer pairs are capable of producing an amplicon greater than or 2000 bases in length.

15. A method for detecting a KIR allele comprising:
(a) detecting one or more amplicons produced by the primer set of claim 5 with a sample having, or suspected of having a KIR allele.

16. The method of claim 15 further comprising:
(b) contacting the primer set of claim 5 with a sample having, or suspected of having a KIR allele, and
(c) producing one or more amplicons of one or more KIR alleles with the primer set if a KIR allele for which a primer set is specific for is present.

17. The method of claim 15, further comprising:
(b) contacting the sample having, or suspected of having, a KIR allele with a primer set that has primer pairs that are capable of producing an amplicon for all presently known KIR alleles.

18. A kit for detecting one or more KIR alleles comprising the primer set of claim 5.

19. A kit for detecting one or more KIR alleles comprising the primer set of claim 7.
